(19) United States
(12) Patent Application Publication
(10) Pub. No.: US 2019/0029637 A1
(43) Pub. Date: Jan. 31, 2019

(54) A WEARABLE SENSOR, AND METHOD, TO MONITOR ANTI-COAGULATION THERAPY

(71) Applicant: The Regents of the University of California, Oakland, CA (US)

(72) Inventors: Jesse Jokerst, San Diego, CA (US); Fang CHEN, San Diego, CA (US); Junxin WANG, San Diego, CA (US)

(21) Appl. No.: 16/072,813

(22) PCT Filed: Jan. 30, 2017
(86) PCT No.: PCT/US2017/015546

§ 371 Date: Jul. 25, 2018

Related U.S. Application Data
(60) Provisional application No. 62/288,829, filed on Jan. 29, 2016.

(51) Int. Cl. A61B 8/06; A61B 5/00; A61K 49/22; A61B 5/145

(52) U.S. Cl. CPC A61B 8/06; A61B 5/0095; A61B 5/6824; A61B 5/14546; A61B 5/4848; A61K 49/221

(57) ABSTRACT

Systems and methods disclosed provide a device that effectively replaces the aPTT test. Chemical sensors are employed that bind to heparin and produce an acoustic signal. The acoustic signal is then used to monitor anti-coagulation therapy, instead of drawing blood, as in the aPTT test. Other quantities of interest can also be measured and monitored.

### CROSS-REFERENCE TO RELATED APPLICATIONS

This application claims benefit of priority of U.S. Provisional Patent Application Ser. No. 62/288,829, filed Jan. 29, 2016, entitled “A WEARABLE SENSOR, AND METHOD, TO MONITOR ANTICOAGULATION THERAPY”, owned by the assignee of the present application and herein incorporated by reference in its entirety.

### FIELD

The invention relates to medical monitoring of blood.

### BACKGROUND

Heparin anticoagulation therapy is a cornerstone of surgical and cardiovascular medicine because of its short half-life, reversible nature, and low cost. However, the same also suffers from a narrow therapeutic window and is the second most common medication error, as it is easy to overdose or underdose heparin. In addition, heparin anticoagulation therapy is challenged by its variable molecular weight, activity, and biodistribution.

For example, such therapy is used prophylactically in patients undergoing angiography, bypass, cannulation, extracorporeal membrane oxygenation, as well as therapeutically in thromboses and cancer. Heparin is used in 15% of pediatric inpatients, and approximately one in seven patients has an adverse event, for an annual morbidity of 140,000. Mortality rates of the diseases treated with heparin are 2-20% and include cerebral sinovenous thrombosis and vascular thromboembolism. Heparin is involved in more medication errors than morphine and vancomycin, with multiple deaths annually. Heparin monitoring is particularly difficult in pediatric populations. Hemorrhages or emboli are common because of inaccurate dosing, dosing/monitoring programs that are designed for adult populations.

Because of these issues, heparin anticoagulation therapy must be monitored very carefully. The current standard of care is the activated partial thromboplastin time (aPTT), in which blood is drawn and tested. However, this in vitro diagnostic tool requires large blood volumes and suffers from long turnaround times, a variable pediatric reference range, and poor correlation to heparin dose/patient performance. Clearly, there is an unmet need for a real-time and non-invasive tool to monitor heparin.

This Background is provided to introduce a brief context for the Summary and Detailed Description that follow. This Background is not intended to be an aid in determining the scope of the claimed subject matter nor be viewed as limiting the claimed subject matter to implementations that solve any or all of the disadvantages or problems presented above.

Photoacoustic imaging is a novel ultrasound technique that uses incident radiation to create thermal expansion—and hence acoustically-detectable pressure differences. Photoacoustic imaging is a light in/sound out technique in contrast to regular ultrasound, which is sound in/sound out. Photoacoustics combines the temporal and spatial resolution of acoustics with the spectral behavior and contrast of optics. Our group² and others³ have applied it to measure tumor/organ size, vascularity, and hypoxic status. Multiple vendors sell commercially available photoacoustic imaging equipment. However, no approach has yet used it to monitor drug levels. Thus, this work is feasible, yet still completely novel.

### SUMMARY

Systems and methods according to present principles meet the needs of the above in several ways. In particular, systems and methods according to present principles include a wearable sensor that can replace or supplement the aPTT test. Chemical sensors are employed that bind to heparin and produce an acoustic signal. The acoustic signal is then monitored, measured, and used to monitor anti-coagulation therapy, instead of drawing blood, as in the aPTT test. A fundamental discovery here is that the clinically-approved small molecule methylene blue—as well as other cationic phenothiazinium dyes—are acoustically silent in the absence of heparin but produce intense, stable, and dose-dependent signal in the presence of heparin.

Although the benefits of this device would be useful in patients with a variety of indications, the field of pediatric coagulation particularly needs new and improved analytical instrumentation to increase the efficacy of heparin monitoring. Systems and methods according to present principles provide a peripheral venous catheter/cannula that measures in vivo heparin levels via a non-invasive ultrasound photoacoustic imaging technique. Using this technique with, e.g., methylene blue, produces a dose-dependent signal in the presence of heparin and is ideal for monitoring drugs, like heparin, with a narrow therapeutic window.

Advantages of the invention may include, in certain embodiments, one or more of the following. Systems and methods according to present principles may, in certain implementations, offer an easy-to-use and accurate noninvasive ultrasound photoacoustic imaging technique. The same may be implemented with a low cost base technology and consumables. The systems and methods allow the monitoring of anti-coagulation therapy without a blood sample, allowing direct control of drug levels and infusion rates without intervention. Virtually instant knowledge of drug levels may be gained, decreasing the time needed to reach the therapeutic window and eliminating venipuncture, peaks, and troughs. Real-time feedback to infusion pumps may be employed to eliminate overdoses and iatrogenic errors. Systems and methods directly measure anti-thrombin activity, as opposed to other techniques, which are indirect surrogate measurements of heparin activity. Because of the use of photoacoustic ultrasound, rather than just in vivo mapping of structure, the acoustic signal actually measures activation of anti-thrombin, thus imaging function, function that could previously only be measured by a venipuncture. Systems and methods according to present principles allow doses to reach a therapeutic window based on real-time data and without invasive blood sampling; moreover the dose is maintained conveniently once sufficient therapy is achieved.

In other advantages, systems and methods according to present principles may employ only one needle, e.g., the one already in place for the IV. While many patients have central ports, drawing large volumes of blood repeatedly over time is difficult because children have a very limited blood pool, and systems and methods disclosed do not require such. Furthermore, prior approaches introduce many opportunities for human error including laboratory, pharmacy, and other medical staff. Thus, the use of this non-invasive, real-time sensor will not only monitor drug levels, but will also eliminate human error. In particular, this may be done via direct communication with the infusion pump for foolproof heparin delivery.

Other advantages will be understood from the description that follows, including the figures and claims.

This Summary is provided to introduce a selection of concepts in a simplified form. The concepts are further described in the Detailed Description section. Elements or steps other than those described in this Summary are possible, and no element or step is necessarily required. This Summary is not intended to identify key features or essential features of the claimed subject matter, nor is it intended for use as an aid in determining the scope of the claimed subject matter. The claimed subject matter is not limited to implementations that solve any or all disadvantages noted in any part of this disclosure.

### BRIEF DESCRIPTION OF THE DRAWINGS

FIG. 1 illustrates two exemplary systems, shown for convenience on opposite sides of a system to be measured, for real-time photoacoustic anti-coagulation monitoring, including for terminating heparin therapy in the event of an overdose, one implementation on the left side of the figure and a second implementation on the right side of the figure.

FIG. 2 illustrates additional details of a measurement system according to present principles.

FIG. 3 illustrates additional details of a photoacoustic measurement device according to present principles.

FIGS. 4 and 5 illustrate schematically other implementations of the present systems and methods according to present principles.

FIGS. 6A-6C illustrate aspects of a photoacoustic signal in whole human blood.

FIGS. 7A-7C illustrate how the photoacoustic signal changes as a function of time and dosing with heparin and protamine sulfate, as well as how the systems and methods according to present principles may be employed with low molecular weight heparin (LMWH). Protamine sulfate is a known heparin antagonist.

FIG. 8 illustrates how heparin and methylene blue may be mixed in blood and imaged with a photoacoustic transducer.

FIG. 9 illustrates more quantitative results of the mixture of FIG. 8 and in particular shows photoacoustic (PA) intensity depending on heparin concentration.

FIGS. 10 and 11 illustrate additional quantities associated with heparin and methylene blue mixed in blood, including a photoacoustic intensity versus heparin concentration and the correlation between active partial thromboplastin time and the photoacoustic intensity. In these and other figures the following acronyms are used: photoacoustic (PA), methylene blue (MB), heparin (Hep), and protamine (Prot). FIGS. 6A, 8, and 10 show similar experiments. Different heparin sensitivities (detection limits) can be achieved by modulating the concentration of phenothiazium dye and gain of the photoacoustic detector.

FIGS. 12 and 13 illustrate how the photoacoustic signal is reversible upon the addition of protamine to the mixture of heparin and methylene blue in PBS.

FIGS. 14-16 illustrate photoacoustic signal measurements, quantitative values of the photoacoustic signal, and activated partial thromboplastin time, when injected into a mouse model according to systems and methods according to present principles.

FIG. 17 is a picture of a catheter treated with a methylene blue dye.

FIGS. 18(A)-18(C) illustrate schematically the binding of the dye to the catheter of FIG. 17.

FIGS. 19(A)-19(D) illustrate alternative types of implementations.

FIGS. 20(A)-20(D) are images of portions of catheters treated with a methylene blue dye in combination with nanoparticles and a hydrogel as suggested in FIG. 18A.

FIGS. 21A, 21B, and 21C show photoacoustic spectra of 5 wt. % oversulfated chondroitin sulfate (OSCS) (A), dermatan sulfate (DS) (B), and chondroitin sulfate (CS) (C) contaminated heparin (red) in the presence of methylene blue.

Like reference numerals refer to like elements throughout. Elements are not to scale unless otherwise noted.

### DETAILED DESCRIPTION

The following glossary of terms is provided as an indicator of certain terms in this application.

Antagonist: An antagonist is a species that attaches to a receptor but does not activate it. It is in contrast to an agonist, which actually does activate a receptor.
Anticoagulation Therapy: Anticoagulants reduce the ability of the body to form a clot. The most aggressive types of anticoagulation therapy use intravenous drugs and include heparin. Anticoagulation therapy is also known as taking blood thinners. It is used during surgery and to prevent clotting.
Antithrombin: Antithrombin is a naturally occurring material in the blood. It reduces blood from forming a clot. Heparin increases the activity of antithrombin by 10³-fold.
aPTT: The activated partial thromboplastin time is a measure of how quickly the blood clots. This is a blood test that needs blood collection from a vein. The blood is sent to a central lab and hours later the test result is returned to the physician. Also known as the PTT.
Cannula: The portion of an “IV” that is inserted into the blood vessel.
Catheter: A tube inserted into the body. An exemplary catheter is the Foley catheter placed into the urinary bladder. Catheters can also be placed into veins or arteries to deliver medications or to allow blood samples to be collected.
Coagulation: The process of blood clotting.
Correlation Coefficient: A measure of how well two variables correspond to each other. Conjugate: A chemical product that results from two species reacting together.
Continuous Glucose Monitor: An implantable device that helps maintain constant glucose levels by automatically dosing patients with insulin as a function of sugar levels.
Contralateral: Meaning on the other side, i.e., “I use my right foot for the gas pedal and the contralateral foot to operate the clutch.”
Deheparinization: The process of counteracting heparin with an antagonist such as protamine.
Embolism: A blood clot traveling through the vein or artery. When the clot blocks blood flow, the area being serviced by the blocked vessel will die. This includes strokes and heart attacks.
Extracorporeal Membrane Oxygenation: Also known as ECMO. This procedure adds oxygen to the blood of children who have injured lungs or circulatory systems.
Flow Chamber: A device used to simulate in vivo blood flow under laboratory conditions. It involves tubing and a pump—these are made in the lab to save costs although commercial systems are available.
Glycosaminoglycan: Polymeric saccharides found widely in mammalian tissue including cartilage and keratin.
Hemorrhage: This is a bleeding event. Hemorrhages involved uncontrolled blood loss from an artery or vein. The result can be coma or even death.
Hemostasis: Hemostasis is the process of stopping blood flow. It involves the creation of a blood clot. The hemostatic system involves many cofactors.
Heparin: Heparin is a polymeric saccharide, i.e., many sugar groups linked together. It has a high negative charge density and is an injectable anticoagulant. Heparin is produced from the intestines of swine during butchering. It is widely used for numerous applications in surgery and cardiovascular disease.
Iatrogenic: Iatrogenic disease is an illness caused during the careless practice of medicine.
I.V. or i.v.: A tube going into a patient's bloodstream that is connected to a bag of medicine hanging on a pole. A pump may or may not be used to control the rate at which this medicine is delivered.
Infusion Pump: These are the pumps that are attached to IVs. The pump moves the medication through the cannula or catheter into the blood stream. Some infusion pumps can be controlled remotely or by the patient. Infusion pumps are sometimes called IV pumps, and may be controlled by a feedback signal, e.g., from a measurement system.
Infrared: The region of the electromagnetic spectrum with energy lower than the visible region, including wavelengths of ˜700-1000 nm.
Overdose or Overshoot: To give a patient too much of a drug.
PBS: Phosphate-buffered saline. A standard biological buffer with pH 7.4.
PEG: Polyethylene glycol is a very common biomedical polymer. PEG is used to coat biomedical devices and to increase the circulation times of injected drugs and imaging agents.
Pharmacokinetics: Pharmacokinetics is the study of what happens to an injected material after it is introduced into the blood stream. It includes questions of metabolism and excretion.
Phenothiazinium: A class of small molecules containing cyclized sulfur and nitrogen groups. These small molecules are dyes and are brightly colored. They are used in cancer therapy and are antimicrobial products. Phenothiazinium molecules are used in certain examples below to measure heparin.
Phlebotomist: A person trained in collecting blood samples from the vasculature via needles and venipuncture.
Photoacoustic Imaging: Photoacoustic imaging converts incident light energy into pressure differences that can be detected via acoustics. It is a non-invasive imaging modality that allows structural, functional and molecular imaging. The light is absorbed by the target and then heats. As it heats, it swells. This swelling—or thermal expansion—creates a pressure difference that can be detected acoustically and used to create an image or quantitate a material of interest.
Protamine sulfate: This is a drug that reverse the effects of heparin.
Therapeutic Window: The therapeutic window is the area in which a drug is both safe and effective. At too high of dose, the drug may have toxic side effects. At low doses, the drug may not be effective at all. The proper balance is known as the therapeutic window or therapeutic range.
Thromboembolism: A blood clot that forms over time in a vessel. A thromboembolism forms in the vein versus an embolism that is mobile in the circulatory system.
Transducer: A transducer is a tool that both detects and emits ultrasound frequencies. It is used for photoacoustic imaging to collect data.
Underdose: An amount of drug that is below the therapeutic window. This is a dose of drug that will not produce sufficient effects.
Venipuncture: The process of collecting venous blood from the veins of a patient using a needle and evacuated glass tubes (see image).

Systems and methods according to present principles provide a tool to quickly measure the concentration of heparin in both buffer and whole human blood using photoacoustic imaging. Systems and methods employ the response that the photoacoustic signal of the clinically-approved methylene blue dye increases dramatically upon binding to heparin.

Systems and methods according to present principles use acoustic signals for therapeutic drug monitoring. Such acoustic signals offer major advantages over optical and electrical signal monitoring, including, e.g., cost, which is low, good transmission through tissue without scatter or diffusion, lack of ionizing radiation, and high temporal/spatial resolution. Systems and methods further employ the discovery that clinically-approved phenothiazinium dyes such as methylene blue produce dose-dependent photoacoustic signal in the presence of heparin. Various dyes are discussed here which are related to methylene blue, including Azure B, phenothiazinium dye, Azure C, and so on. Such dyes are said to be photoacoustically active, so long as they produce a dose-dependent photoacoustic signal in the presence of (and/or when bonded to) a quantity of interest such as heparin. Various dyes and classes of dyes will be discussed here, and term usage may vary according to context, e.g., dye, methylene blue, phenothiazinium, phenothiazine, or just ‘dye’. Generally, dyes may be employed that include a charged species that interacts with heparin.

In more detail, a non-invasive imaging technique is employed to quantitate heparin. This approach is based on photoacoustic imaging, which is a light-in/sound-out imaging tool, versus the conventional sound-in/sound-out approach in conventional ultrasound. Photoacoustic imaging combines the high spatial and temporal resolution of ultrasound with the spectral behavior and contrast of optics. Photoacoustic imaging can use endogenous contrast such as hemoglobin and melanin as well as exogenous contrast including small molecules like methylene blue and nanoparticles. While photoacoustic flow cytometry has measured emboli based on the absorption change and nanosensors have been reported for therapeutic drug monitoring of lithium, there is no approach to monitoring heparin therapy with acoustics.

### Exemplary Device Description

Systems and methods according to present principles use a photoacoustic system in combination with a photoacoustically active dye. The photoacoustic system employs optical emission to irradiate the target and a receiver (transducer) to receive waves from the excited particles. In more detail, the photoacoustic system employs a transmitter of light to excite heparin, bound to a dye, and an ultrasound transducer to detect the acoustic signals generated by the heparin/dye combination.

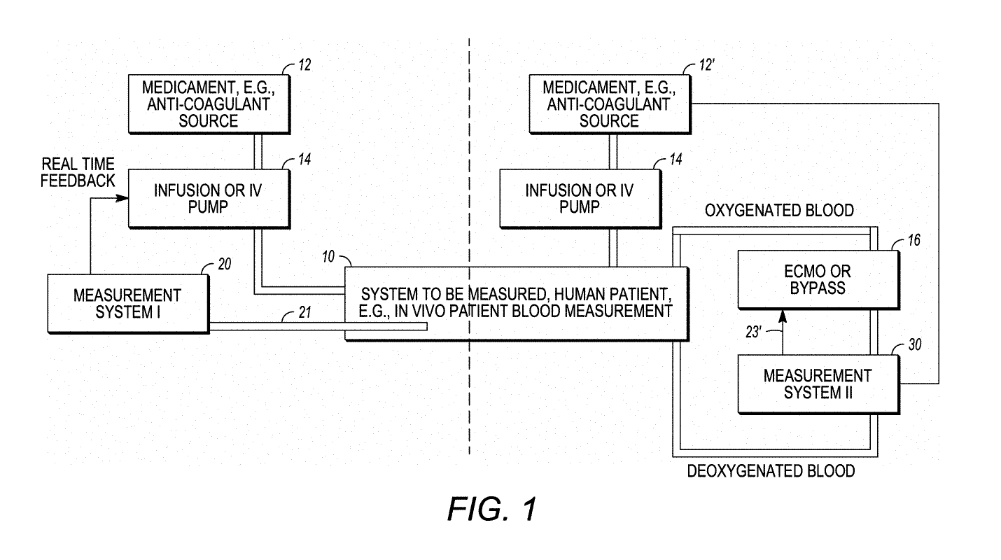

As may be seen in FIG. 1, a catheter-based measurement system I is indicated by element 20 that in one implementation (left side) includes a catheter or other indwelling or implanted probe 21 situated at least partially within a system 10 to be measured. For example, the system 10 may be a human patient. In this implementation, the measurement system 20 has an output that is sent to an infusion or IV pump 14 which receives a medicament such as an anticoagulant from anticoagulant source 12. The IV pump 14 pumps the anticoagulant into the system 10. Measurement system 20 measures via catheter or probe 21 the heparinization level within the system 10, and provide signals to the IV pump 14 so as to keep the level of heparinization within a therapeutic window.

Similarly, in an ECMO or bypass situation, shown on the right-hand side of FIG. 1, controlled by a machine 16, a second type of measurement system II, indicated by reference numeral 30, is indicated which can measure the level of heparinization extracorporeally. As indicated by connection 23, the measurement system 30 can similarly control an IV pump 14′ which is coupled to an anticoagulant source 12′, or alternatively the measurement system 30 can control an aspect of the ECMO or bypass machine 16 via connection 23′. It will be understood that other alternatives are possible, including the use of telemetry that communicates with external devices or stations, e.g., a nursing station.

In any of the implementations, heparinization values determined by the measurement system may be employed to keep the level of heparinization within a therapeutic window. If the level gets too high, i.e., approaches a predetermined high threshold level, delivery of heparin may be reduced or stopped. Similarly, if the level gets too low, i.e., approaches a predetermined low threshold level, then delivery of heparin may be increased or started.

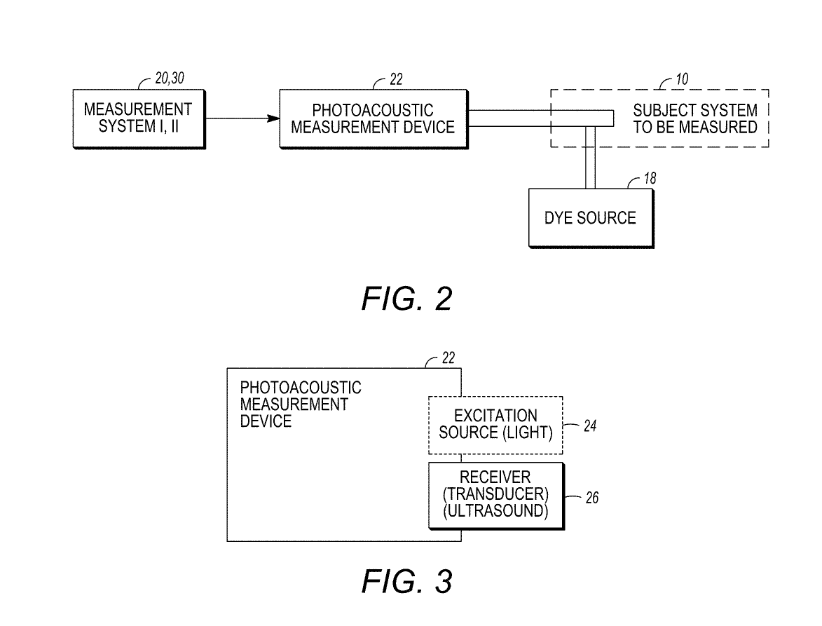

Certain additional details of the measurement system 20, 30 are shown in FIG. 2. In this figure, the system is shown to include a photoacoustic measurement device 22 and a dye source 18. As will be described, the dye source can generally provide dye into the bloodstream or can provide the dye in a localized manner, i.e., such that the dye remains in the vicinity of the photoacoustic measurement device 22. It will be understood that the dye source can be immoblized at the location of interest or could be delivered to the location of interest.

Referring in addition to FIG. 3, the photoacoustic measurement device 22 includes an excitation source 24, which is generally an optical wave such as light, and a receiver or transducer 26, which is generally configured to receive and measure ultrasonic or ultrasound waves. Exemplary excitation sources may include LED light sources, as well as optical parametric oscillator (OPO) lasers. In one implementation, an OPO laser was used that was tunable from 680-970 nm. A LED exccitation source at 690 nm was also used. These lasers both used energies of less than 20 mJ/cm².

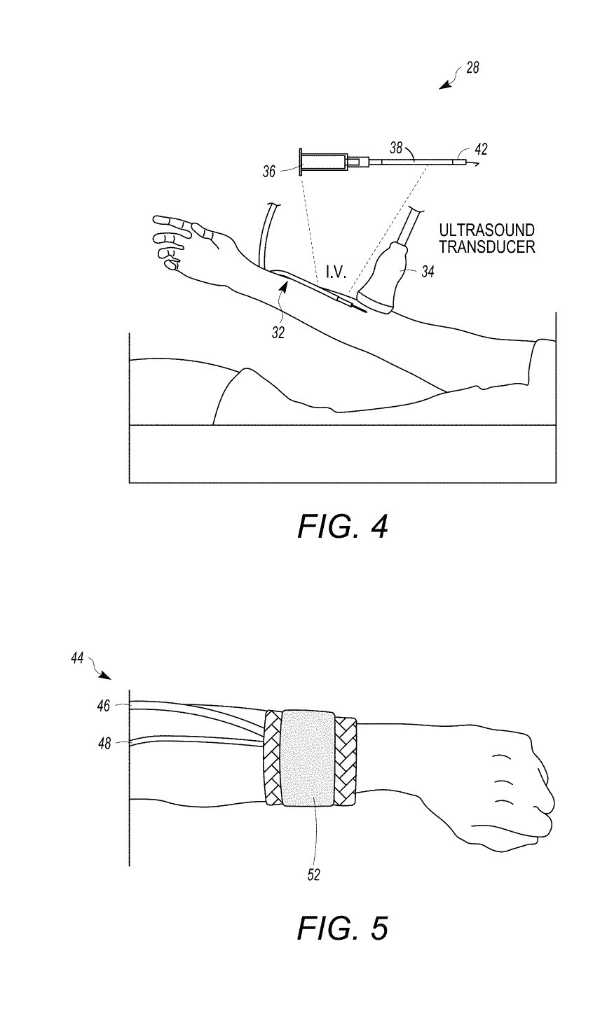

FIG. 4 illustrates additional implementations for real-time sensing of heparin. In more examples, a system 28 is shown including a catheter 32 and an ultrasound transducer 34. The catheter 32 may be situated within the vein of a subject patient. The catheter 32 includes drug sensitive cladding 38, as well as a luer-lock adapter 36. The distal tip of the catheter is shown as tip 42. In this case, the drug-sensitive cladding 38 includes the dye which enables the drug sensitivity, i.e., is sensitive to the level of heparinization, and the same emits an acoustic signal that can be picked up by the ultrasound transducer 34, the acoustic signal related to the level of heparinization. In one implementation, where an OPO laser is employed, the optics from the laser can be integrated into the transducer, such that the portion of the body adjacent the transducer can be effectively illuminated, e.g., either directly by the laser or by laser light following an optical path and then illuminating the portion of the body. The optics may also be mounted exterior of the transducer. A general requirement is that the area or volume in which the dye is located should be able to be illuminated by the light source. In one implementation, this is accomplished by ensuring that the surface of the skin can be illuminated by the light source.

FIG. 5 illustrates an alternative implementation 44. In this case, the IV tubing is collectively indicated by element 46, and the patient is fitted with a wearable transducer 52. Photoacoustic fibers 48 are shown as being usable to provide excitation and measurement functionality via a wearable cuff. In this concept, LED excitation sources and transducers are embedded in the cuff, and the cuff is then secured to the patient.

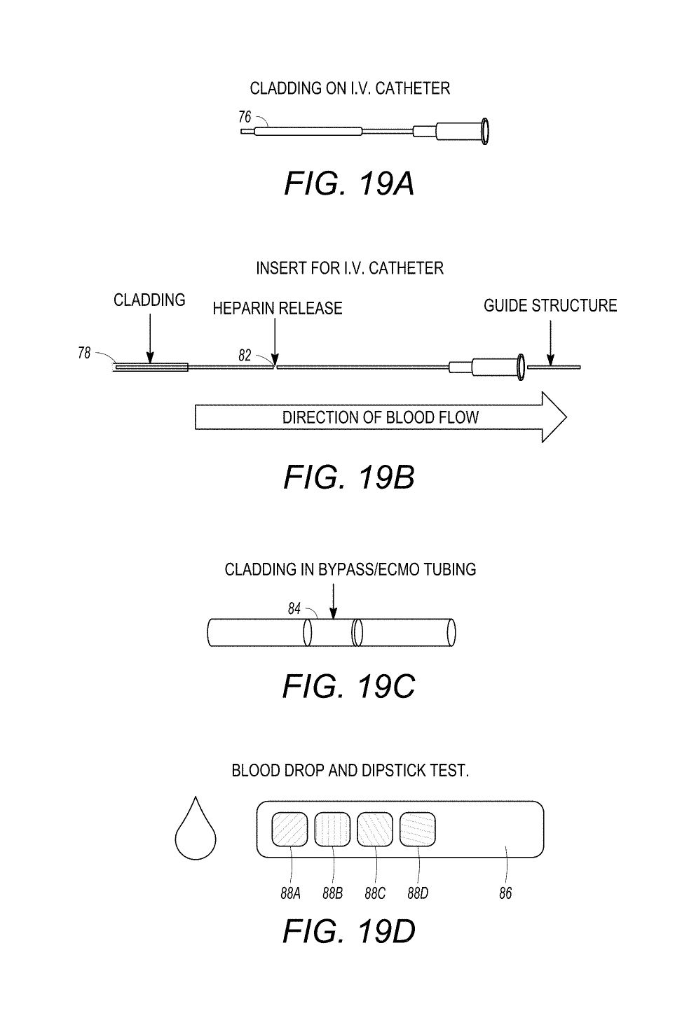

In the above implementations, venous catheters may be refashioned as “smart catheters” and may work in tandem with wearable ultrasound transducers. In particular, a catheter with cladding in which the dye is situated may be inserted into the venous catheter for measurement purposes, and a wearable ultrasound transducer may be disposed outside of, but coupled to, the patient. See, e.g., FIG. 19(B).

Practical uses include telemetry or the surgical ward, reducing or eliminating the need for venipuncture or pharmacy/laboratory intervention. In another approach, the sensor may be built into the perfusion circuit (see the right side of FIG. 1) for real-time monitoring of clotting time during ECMO or bypass and to confirm protamine deheparinization.

Regarding the dye used, it has been found that the non-toxic small molecule phenothiazinium dye binds to heparin and has low photoacoustic activity, but produces a robust, stable, and dose-dependent photoacoustic signal in the presence of heparin. This dye may thus be employed as part of the implantable sensing device that works in tandem with a wearable ultrasound transducer to monitor heparin levels. The sensor may further be employed, e.g., in tandem with smart infusion pumps, to automatically control and in some cases to terminate IV heparin therapy in the event of an overdose (see, e.g., the left-hand side of FIG. 1).

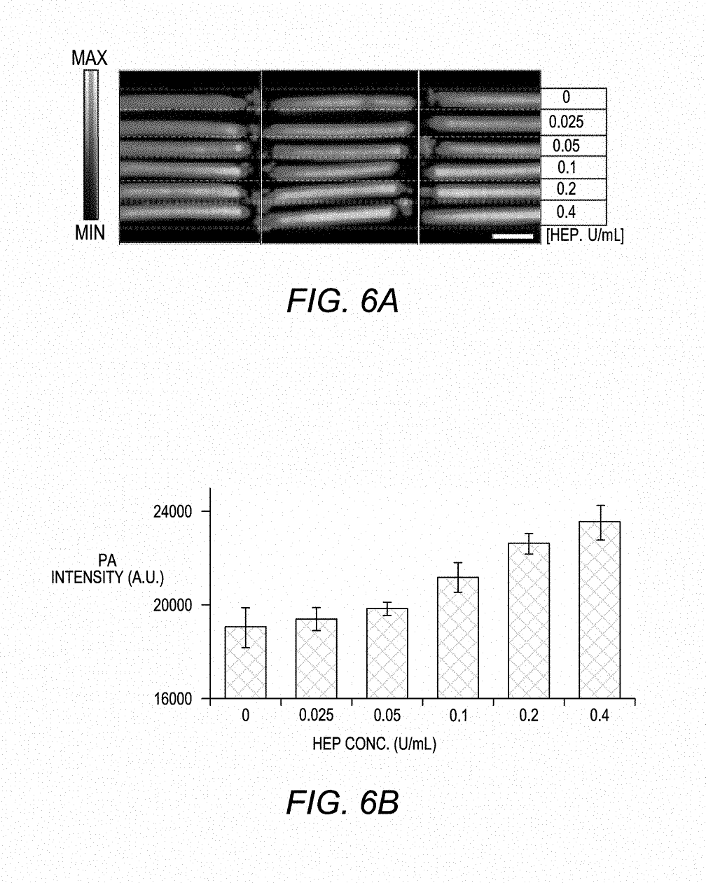

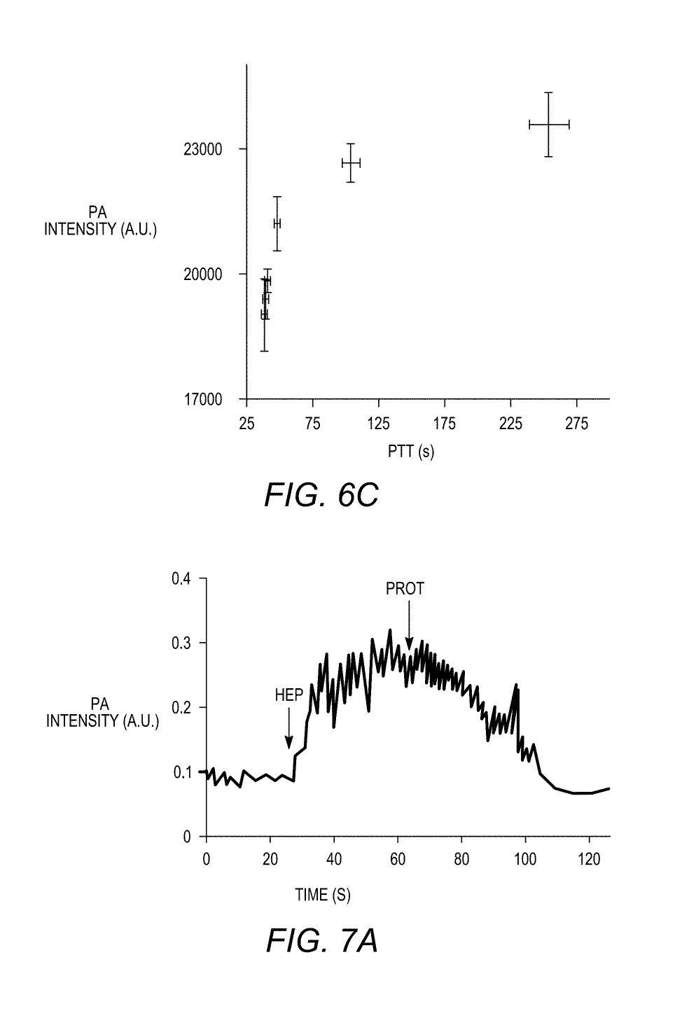

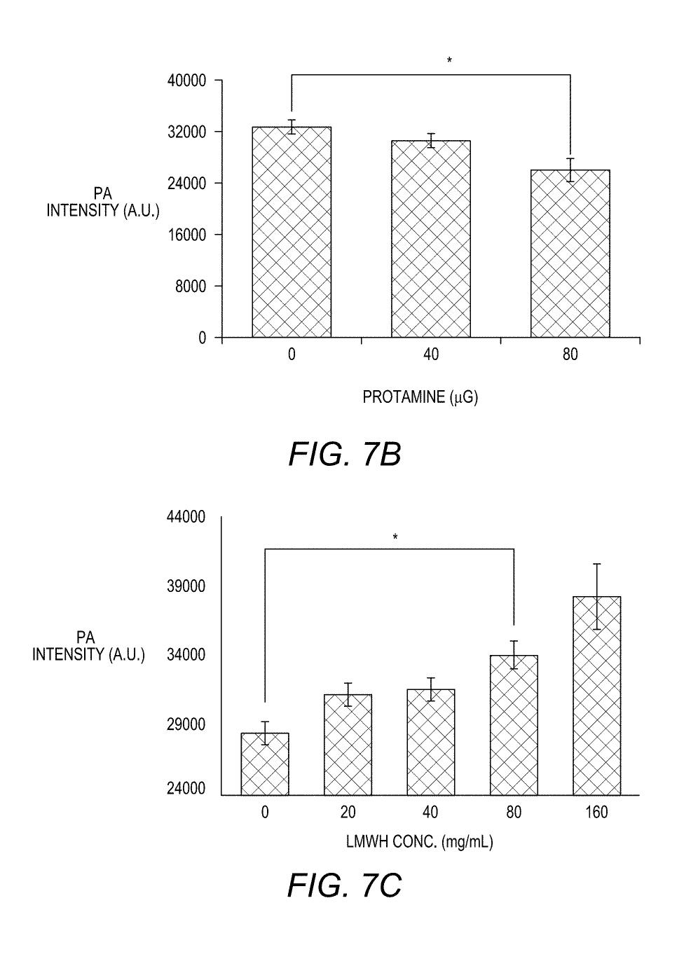

FIGS. 6A-6C and 7A-7C illustrate photoacoustic signal in whole human blood. In more detail, FIG. 6A illustrates a photoacoustic image of plastic tubing containing human blood with 0.8 mM methylene blue and increasing concentrations of heparin with 0.8 mM methylene blue (n=3 replicates). The scale bar in FIG. 6A is 3 mm. FIG. 6B quantitates the three replicates in FIG. 6A, and the detection limit of heparin is 0.28 U/mL. FIG. 6C plots the photoacoustic intensity versus aPTT. The Pearson's correlation coefficient (r) was 0.86; p<0.05. FIG. 7A shows the photoacoustic signal changes of a blood/methylene blue mixture as a function of time when heparin (at 27 s) and protamine (at 68 s) were added. FIG. 7B shows that the heparin signal is reversible in whole blood with titration of protamine, which is the known heparin antagonist. FIG. 7C illustrates that the approach may also be employed with increasing concentrations of low molecular weight heparin (LMWH) in methylene blue-doped human blood. The lowest concentration of LMWH that could be detected in blood is 72 μg/mL. Error bars represent the standard error. *: p<0.01.

FIGS. 6 and 7 thus validate the technique by showing clear measurable signals when methylene blue is added to blood, and how the addition of heparin increases the signal even further. In addition, good correlation is seen between clotting time and photoacoustic signal as measured by aPTT and the photoacoustic technique, suggesting that the measured imaging data is representative of actual clotting time, as aPTT is the current gold standard method. The figures also show that protamine sulfate reversal could also be performed and that similar signal enhancements could also be seen with LMWH.

Methylene blue is already used to visualize sentinel lymph nodes and has broad absorption peaks between 500 and 700 nm, but only nominal photoacoustic signal in the optical window typically used for imaging (680-900 nm; sensitivity of 7.1 μM at 680 nm). When electrostatically coupled to heparin, however, the absorption and color of methylene blue changes. Somewhat surprisingly, this heparin complexation also produces an intense increase in photoacoustic signal. Systems and methods according to present principles optimize the methylene blue concentration as a function of signal. For example, 6.4 U/mL heparin and 0.6 mM methylene blue produced the best contrast when added to heparin versus free methylene blue only in buffer.

Linear regression shows a strong correlation between photoacoustic signal and heparin concentration from 0 to 6.4 U/mL (exponential linear regression R2>0.98) with a detection limit of 14.2 mU/mL. Spectral photoacoustic imaging of these samples showed a maximal intensity absorbance at 680 nm with a characteristic peak near 710 nm and a 30-fold increase in photoacoustic signal with 6.4 U/mL heparin at 710 nm excitation. Heparin concentrations over 10 U/mL show decreased signal.

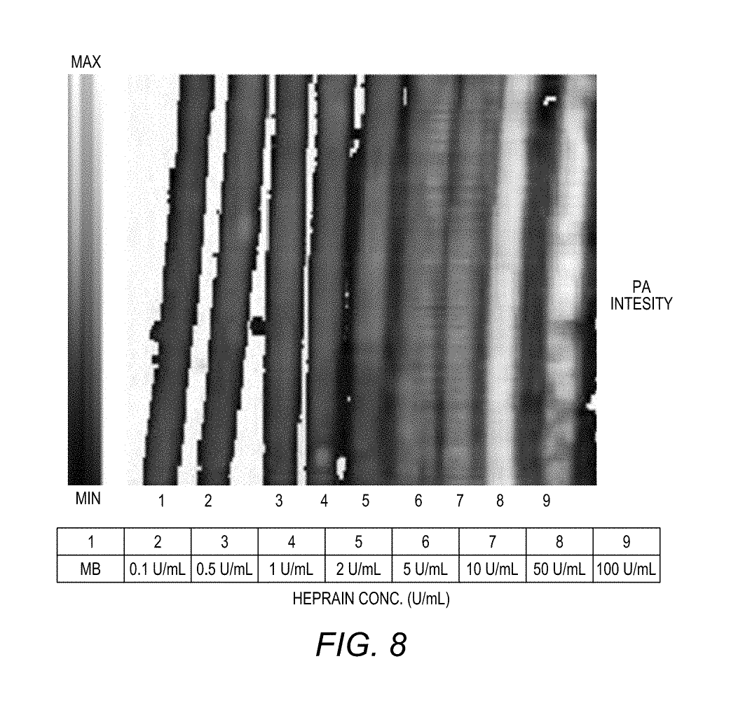

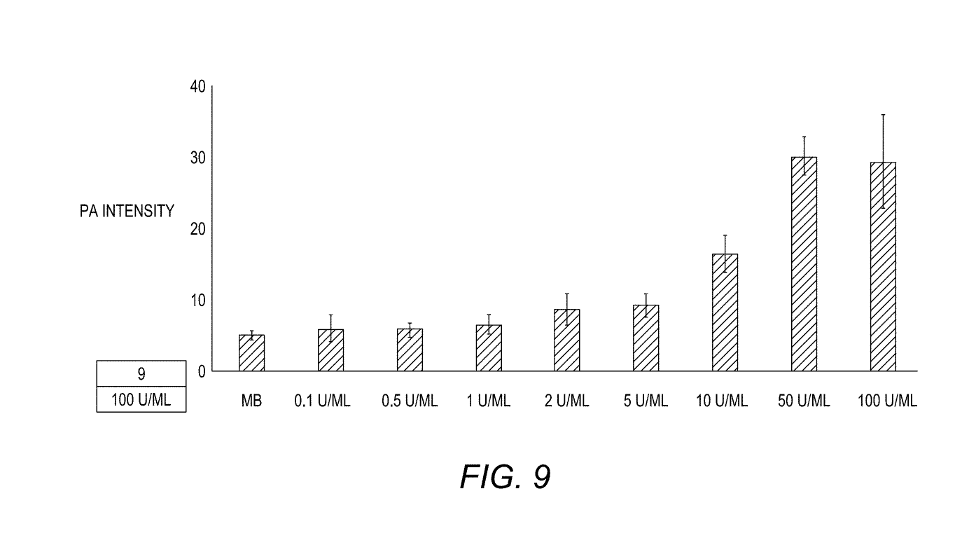

FIGS. 8 and 9 illustrate more quantitative results of an exemplary mixture of the type described, again indicating the consistency of the rise of signal with respect to heparin level.

The capacity of phenothiazinium dye to quantitate heparin has been measured as described above. However, this small molecule will passively diffuse once implanted into the body. Thus, nanoparticles may be employed to immobilize the dye into a specific region of the body. For example, the dye may be incorporated into poly(lactic-co-glycolic acid) nanoparticles or polymeric micelles. These nanoparticles allow the heparin access to the dye while minimizing dye clearance during the imaging. In one implementation, it is believed that it may be appropriate to encapsulate 1 mg of phenothiazinium dye per mL of nanoparticle volume, as this has been shown to produce optimal photoacoustic signal in other preliminary data. More generally, 0.5-5 mg of phenothiazinium dye per mL of nanoparticle volume may be employed. Concentrations may also be expressed in molar units. After the nanoparticles are prepared, they may be stabilized via freeze-drying for long-term storage. Increasing concentrations of heparin may be added and the signal may be monitored as a function of concentration. In some implementations, planar substrates may be employed rather than nanoparticles.

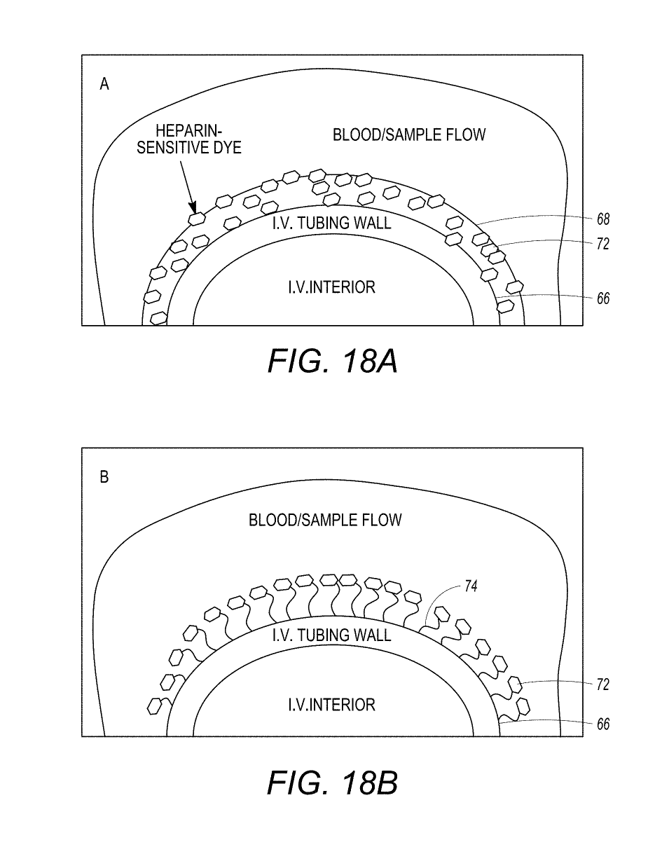

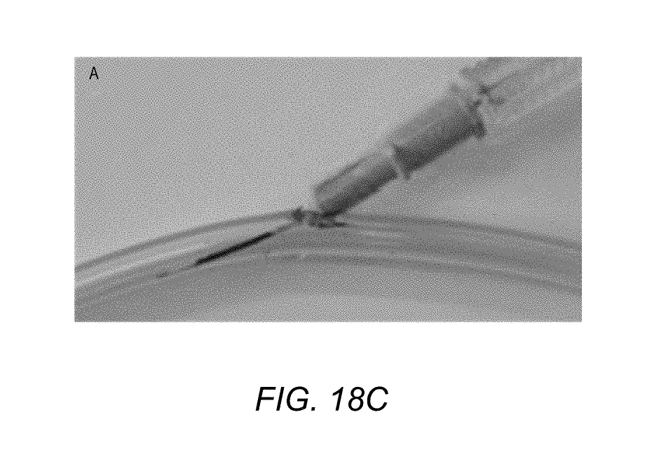

In more detail, to encapsulate the dye on a usable substrate, systems and methods according to present principles employed a hybrid of agar gel and silica nanoparticles that could be coated on the surface of a catheter as suggested in FIG. 18A. Mixing methylene blue with agarose resulted in quick release of 40% of the dye. While methylene blue could be stably bound to Stober silica nanoparticles (SSNP), there was no photoacoustic response when heparin was added. The surface charge of these nanoparticle is −23 mV, which is too negative to allow association between the nanoparticle-bound dye and negatively charged heparin. Therefore, the charge of the particle was changed to produce a material that was both stable and responsive to heparin. To tune the nanoparticles, the same were treated with (3-mercaptopropyl) trimethoxysilane (MPTMS) to adjust the zeta potential to −15 mV (SSNP-SH). This material was used with agar gel to create a coating on a catheter tube.

Absorption data showed that the hybrid material has a methylene blue concentration of 1.5 mM with low methylene blue release (<10%) after 40 min incubation in PBS. More importantly, the methylene blue on these modified nanoparticles remain responsive to heparin with 51% more photoacoustic signal at 10 U/mL heparin than PBS for 1 h incubation. The signal enhancement was stable after constant imaging at 680 nm for 9 min (RSD<4%) This system was also reversible. When the 50 U/mL-treated device was reversed with protamine (8 mg/mL), the signal decreased by 42% versus no protamine treatment. The higher signal of the heparin-treated hybrid versus PBS-treated hybrid is likely due to interactions between methylene blue and heparin, which increases the photoacoustic signal and decreases diffusion of this complex and free methylene blue into solution.

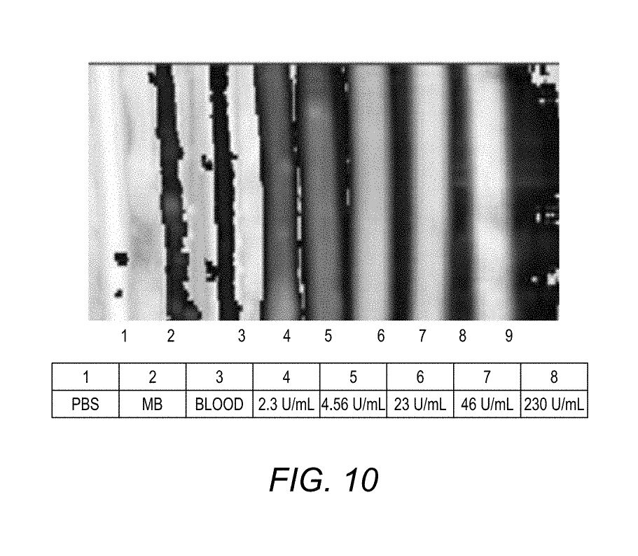

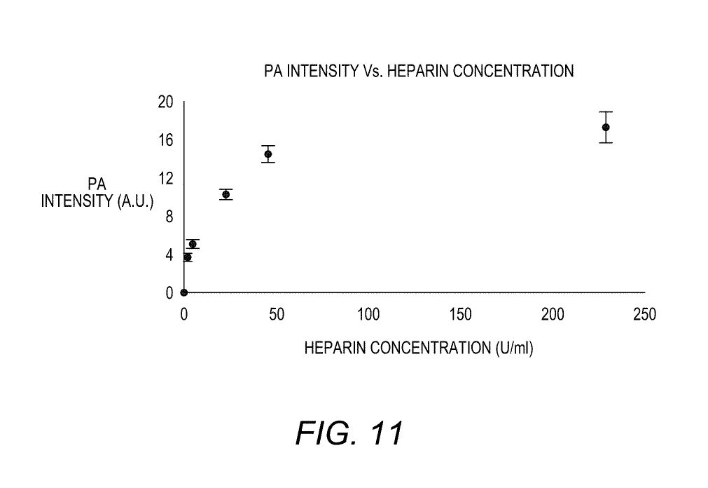

FIGS. 10 and 11 illustrate additional quantities associated with heparin and methylene blue mixed in blood. In more detail, FIG. 10 illustrates various concentrations and FIG. 11 illustrates photoacoustic intensity versus heparin concentration.

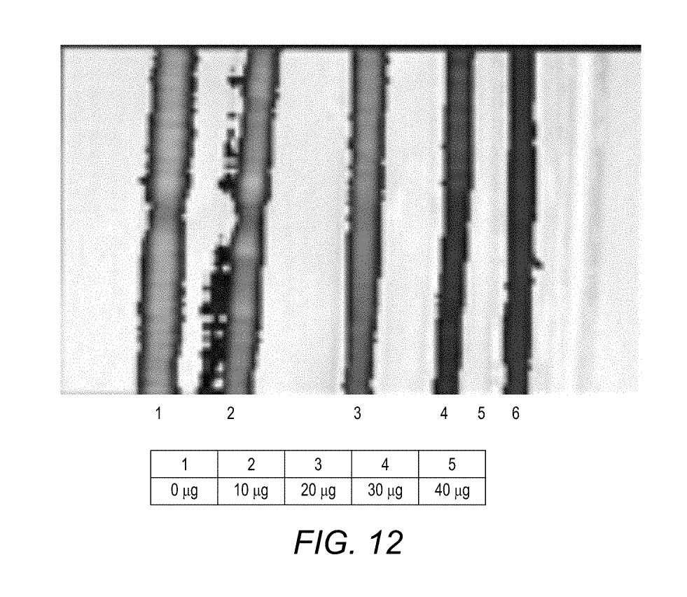

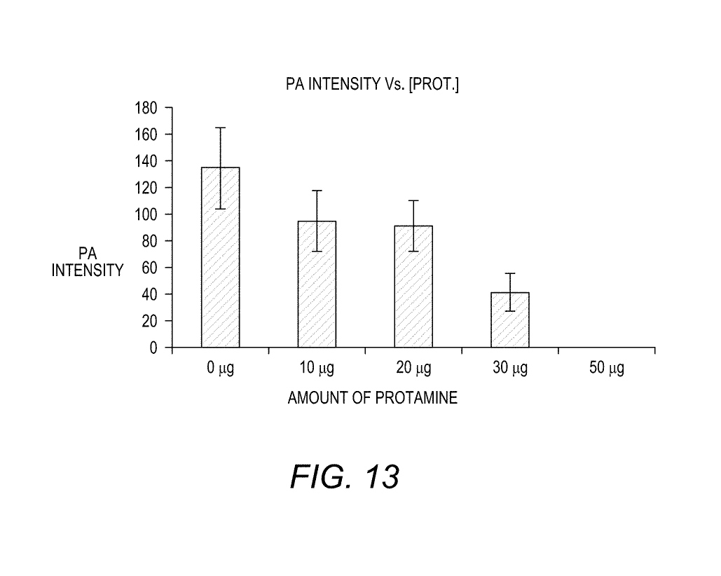

Importantly, the approach is reversible—the signal and color change is reversible when the phenothiazinium dye/heparin complex is treated with protamine sulfate (the clinically used-antidote to heparin overdose). In more detail, FIGS. 12 and 13 illustrate how the photoacoustic signal is reversible upon the addition of protamine to the mixture of heparin and methylene blue. In particular, the figures show the photoacoustic signal versus increasing amounts of protamine in the mixture that contains heparin of five units. Groups 1-5 correspond to the protamine amounts of zero, 10 μg, 20 μg, 30 μg, 40 μg, and 50 μg, respectively. FIG. 13 illustrates more quantitatively the photoacoustic signal, and in particular shows how the signal can be reduced substantially with administration of protamine.

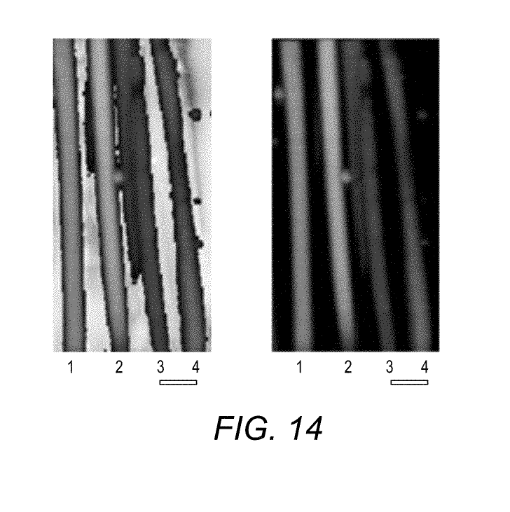

FIGS. 14-16 illustrates how the approach can be used in living subjects. In this experiment, four mice were injected with either 10 units per milliliter of heparin or zero units per milliliter of heparin. 30 minutes later, they were injected with methylene blue. 30 minutes after that, their blood was collected and imaged using systems and methods according to present principles. The remaining blood was analyzed with a conventional approach, i.e., aPTT.

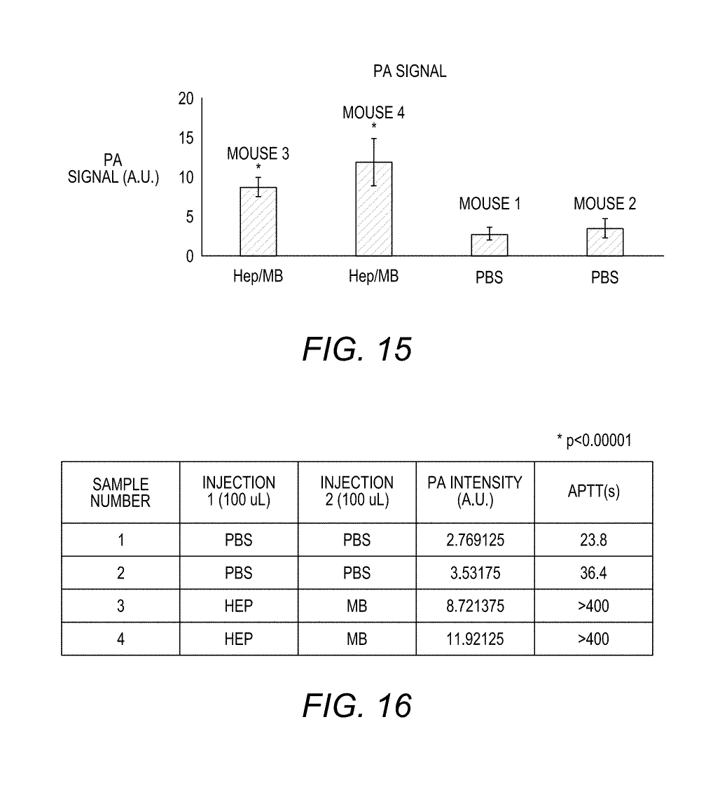

In more detail, FIG. 14 illustrates the photoacoustic signal measurement of blood drawn from mice after injection with methylene blue and heparin. Mouse 1 and 2 were injected with heparin followed by methylene blue. The concentration of heparin and methylene blue in the mice was 10 units per milliliter and 2.5 mM, respectively. Mouse 3 and 4 were injected with 200 mL of PBS. FIG. 15 illustrates quantitatively the photoacoustic signal. FIG. 16 illustrates the activated partial thromboplastin time of samples 1-4.

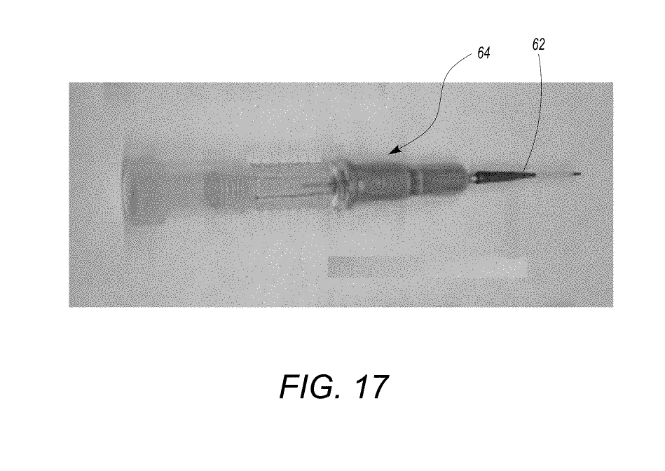

FIGS. 17-18 illustrates how this sensitive methylene blue dye may be placed onto a catheter that could be immobilized in the bloodstream. FIG. 17 images the final result with a heparin sensitive cladding 62 seen on catheter 64. To construct this implementation, a venous catheter was dipped in a mold containing agarose and methylene blue. The agar was allowed to congeal around the catheter.

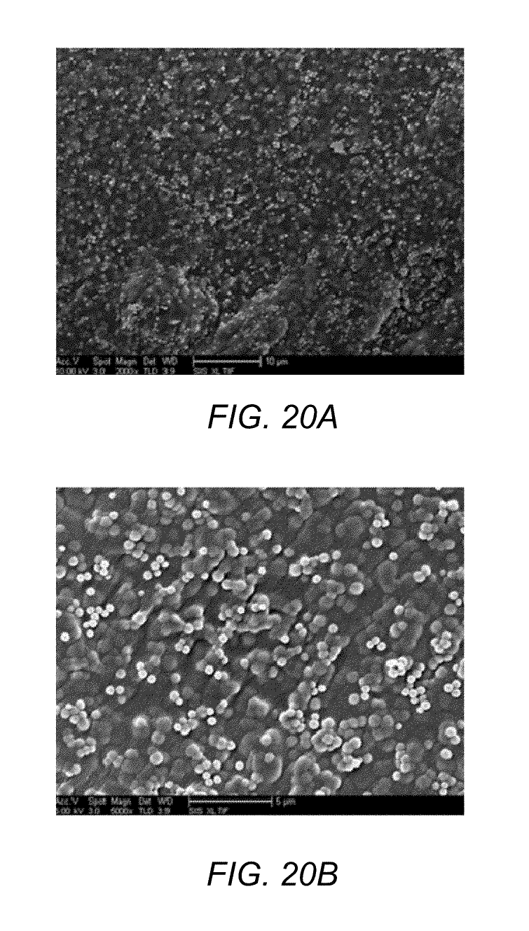

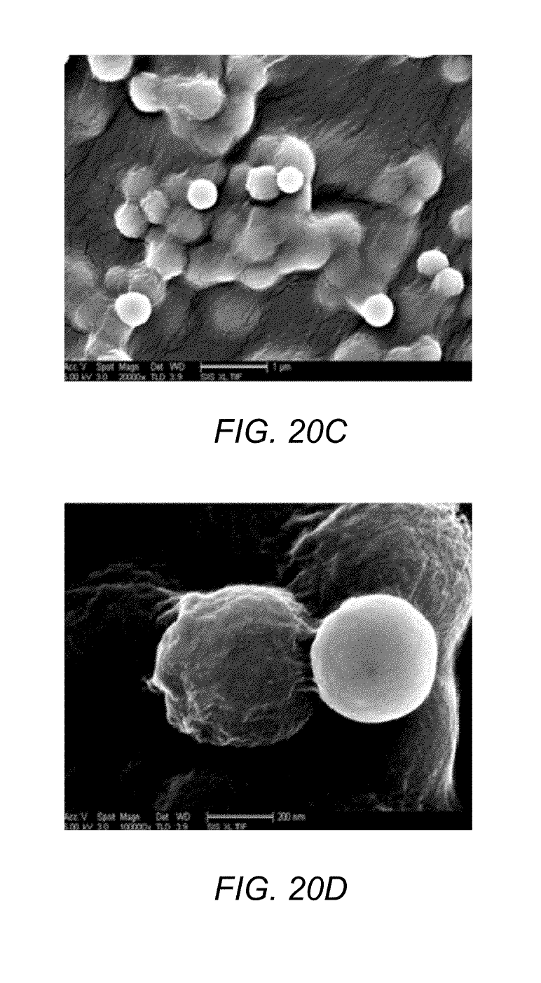

The mechanism of attachment is shown in FIG. 18. In FIG. 18(A), dye (blue, elements 72) is embedded in a gel 68 on the IV tubing. In FIG. 18(B), the dye is covalently bound (bonds 74) to the tubing, with the result seen in FIG. 18(C). Nanoparticles may also be doped into the agar/methylene blue mixture to control the sensitivity of the approach, as may be seen by the electron microscopy images of FIGS. 20(A)-20(D). The hydrophobicity and roughness of the device surface may be tuned to minimize biofilm formation. Antibacterial coatings may also be employed.

FIGS. 19(A)-19(D) illustrates various alternative designs, i.e., variations on designs described above. FIG. 19(A) illustrates a heparin sensitive region 76, accomplished by methods noted above including embedding the photoacoustic dye within the cladding on the catheter. FIG. 19(B) illustrates how an IV insert could be employed to move the heparin sensitive region 78 away from the drug delivery region 82 to reduce locally high concentrations. FIG. 19(C) illustrates dye embedded (cladding region 84) in blood tubing during perfusion or ECMO. Finally, FIG. 19(D) illustrates an external “dipstick” for point of care testing.

In more detail, with respect to FIG. 19(B), the catheter employs a heparin-sensitive cladding 78 on an extension that is sufficiently upstream from the drug release region 82 to minimize interference from the high local concentrations of heparin at the end of the catheter. The transducer would then be worn several inches upstream of the IV. With respect to FIG. 19(C), the dye could be immobilized inside of perfusion tubing (region 84) for use in the operating suite or during ECMO, further minimizing risks. With respect to FIG. 19(D), a finger stick approach may be employed in which the dye is immobilized on a test strip 86 and evaluated with an external photoacoustic reader. Regions 88a-88d with varying levels of dye as well as regions for positive and negative controls may then be employed to quantify the level of heparin. This may be used with a bedside photoacoustic device.

Systems and methods according to present principles provide an analog to continuous glucose monitoring or pulse oximetry, but for anticoagulants. In this way, intravenous catheters may be employed to not only deliver drugs, but also to monitor drug activity. Other applications include digoxin and phenytoin. In more detail, digoxin is a common drug used to treat atrial fibrillation and heart failure with a narrow therapeutic window. Its chemical structure has multiple glycosidic functional groups and bonds. These chemical structures have unique charge distributions that could be exploited via acid dyes including aniline derivatives for photoacoustic imaging of digoxin levels in vivo. There are also many aromatic chelators that can be used to detect metal cations via photoacoustic imaging. For example, instant measurement of electrolytes and Ca, Mg, and Cu is also possible. Most importantly, photoacoustic imaging is spectral. Thus, a wearable sensor may be employed that monitors multiple drugs concurrently via different excitation wavelengths, e.g., heparin at 680 nm, digoxin at 750 nm, and calcium at 950 nm.

Variations will be understood. For example, while methylene blue has been described, other potential phenothiazinium species may include: toluidine blue, phenothiazinium dye, methyl methylene blue, dimethyl methylene blue, azure B, azure C, thionin, methylene violet, and the like. While the systems and methods disclosed above relate primarily to levels of heparin, LMWH levels may also be measured, which can currently only be monitored with anti-Factor Xa ELISA testing. We refer to these species as dyes or phenothiazinium dyes or phenothiazines here. It is also understood that chemical variations of these species may be expected. In one conception, polymeric versions of these dyes may be used on the surface of a device. In another conception, pendant chemical groups may be added to the molecule to increase or decrease the charge and increase or decrease the photoacoustic signal upon interacting with heparin.

As mentioned above, systems and methods according to present principles may be employed in the development of a bedside unit, where a finger prick sample is used along with a bedside photoacoustic analyzer. A more sophisticated unit may employ a wearable, heparin-sensitive catheter that not only delivers heparin but also monitors clotting time.

The strengths of this approach include the rapid turnaround time, excellent sensitivity, good correlation to the aPTT, and flexibility with both heparin and LMWH. While the aPTT is the gold standard for monitoring anticoagulation, it suffers from long turnaround times and limited utility with LMWH. In contrast, it only takes 0.2 s for photoacoustic imaging to acquire one data point (5 Hz imaging) and can be used with both unfractionated and LMWH, although more than one frame might be needed for an accurate and stable signal. This approach could also leverage spectral or ratiometric imaging to decouple the heparin-specific signal from other sources of photoacoustic signal.

Limitations to this approach include different PA background due to hydration state, oxygenation, body fat content, and location of the catheter. However, the change in signal versus baseline may be monitored as a function of heparin therapy to control for these variations.

What has been shown are systems and methods which employ methylene blue which has a significant and dose-dependent increase in photoacoustic signal in the presence of both heparin and LMWH. This signal was validated in both buffer and whole blood with good correlation to the gold standard aPTT. The signal was reversible with protamine sulfate deheparinization. Photoacoustic imaging is a real time technique, and methylene blue is a FDA-approved dye. Thus, such systems and methods provide for real-time monitoring of anticoagulation therapy to quickly titrate the patient into the therapeutic window. This has potentially profound implications because acoustics-based wearable sensors have low costs, good transmission through tissue without scatter/diffusion, no ionizing radiation, and high temporal and spatial resolution, in contrast to many optical or electrical sensors.

In a variation, the systems and methods according to present principles may also be employed in the detection of contaminants in heparin. In particular, and as a particular example, in early 2008, oversulfated chondroitin sulfate (OSCS) was identified by a joint research team as the major contaminant in the supply of heparin that caused over 100 deaths and resulted in a massive recall of heparin from US and European markets.

Heparin quality control involves a coagulation test, but this screen did not detect OSCS because it has a high degree of anti-factor 11a activity. Because the traditional screening methods were unable to differentiate the contaminants from heparin, two compendial test methods for heparin had to be updated—proton nuclear magnetic resonance (1H-NMR) and capillary electrophoresis (CE). 1H-NMR is used to characterize the chemical structure of monosaccharide building block. 1H-NMR and CE can quantitate OSCS in heparin at a concentration as 0.1 wt % and 0.05 wt %, respectively. Strong anion exchange-high performance liquid chromatography (SAX-HPLC) can also been used and has good detection limits (LOD=0.03 wt %) and is convenient. Other sensitive methods for measuring OSCS in heparin include heparin enzyme immunoassay (LOD=0.1 wt %), potentiometric assay (LOD=0.5 wt %), and colorimetric chemosensors (LOD=0.003 wt %). Despite the high sensitivity of these methods, they require a period of time for analysis and the analytes must be dissolved in aqueous solutions. Systems and method according to present principles, using photoacoustic imaging, may be employed to rapidly image the OSCS and dermatan sulfate (DS)—known heparin impurities—in buffer and human whole blood.

As noted above, methylene blue can be used as a photoacoustic probe to measure heparin concentrations. Photoacoustics uses nanosecond laser pulses to generate thermal expansion in targets and measure the induced acoustic waves, where it combines the fast response and high resolution of ultrasound and high contrast from optics. The signal intensity and spectral behavior varies as the chemical structure and electron density changes. While the chemical structure of heparin, OSCS, DS, and chondroitin sulfate (CS) are similar, the DS or CS have different sulfated moieties and OSCS has a higher negative charge because of the highly sulfated moieties.

Thus, the signal intensity at a specific wavelength or the ratiometric intensity at multiple wavelengths can be used to differentiate OSCS contaminated heparin or DS contaminated heparin from pure heparin in the presence of methylene blue. The ratiometric photoacoustics is a robust measurement method because it can eliminate the background noise from different measurements. Thus the technique may be used in the measurements of OSCS or DS contaminated heparin in human whole blood.

Measuring drugs in blood is usually difficult because blood contains many materials that can interfere with signal or generate background signal. However, by adding methylene blue, the photoacoustic signal of blood containing OSCS contaminated heparin or DS contaminated heparin is higher than blood containing pure heparin.

In one exemplary implementation, the photoacoustic imaging system used for measurements is a Vevo 2100 commercial instrument (Visualsonics). The scanner is equipped with a 21 MHz-centered transducer (LZ250) and a Q-switched Nd:YAG laser. The laser uses an optical parameteric oscillator and second harmonic generator to generate a tunable laser between 680 and 970 nm with a 1 nm step size and a pulse of 4 to 6 ns at 20 Hz. The peak energy is 45±5 mJ at 20 Hz at the source. The full field-of-view is 14-23 mm wide. The acquisition rate is 5 frames per second. The samples were prepared in small tubing fixed in a self-developed phantom holder which was aligned at ˜10 mm under the transducer. The laser energy was calibrated and optimized using the build-in energy power meter and software before measurements. 100% laser energy was used with 21 MHz frequency and the gain was optimized from 10-40 dB to obtain 3D scans at 680-850 nm as well as photoacoustic spectra from 680 to 850 nm.

1×PBS solution was prepared via dissolving one PBS tablet into 200 mL deionized water. The 2 mM methylene blue was prepared fresh by dissolving reagent-grade powder in PBS followed by sonication for 30 minutes at 40 C and then filtering through 0.22 μm. The 0.4 mM methylene blue was prepared by dissolving 2 mM methylene blue with PBS. A batch of OSCS/heparin mixture that simulates OSCS contaminated heparin was prepared at the following steps: 100 μL of 50, 25, 5, 2.5, 0.5, 0.25 μg/mL OSCS, and PBS was added into 100 uL 1000 μg/mL heparin prepared in PBS solution, respectively. The working concentration of OSCS in the above solutions was 25, 12.5, 2.5, 1.25, 0.25, 0.125, 0.025, and 0 μg/mL OSCS, respectively, and the working concentration of heparin is 500 μg/mL. In other words, the heparin solution was contaminated by 5, 2.5, 0.5, 0.25, 0.5, 0.25, 0.05, 0.025, and 0%. The contrast signal of methylene blue/heparin mixture was optimized at the combination of 0.4 mM methylene blue and 50 μs/mL. 20 μL “contaminated heparin” was added into 180 μL of 0.4 mM methylene blue. Two controls were included; methylene blue only and chondroitin only. They were prepared by adding 20 μL PBS and 50 μg/mL OSCS in 180 μL 0.4 mM methylene blue, respectively. The simulated dermatan sulfate and chondroitin sulfate contaminated heparin were prepared via a similar method by replacing OSCS with dermatan sulfate or chondroitin sulfate.

Data analysis were performed using the scanner's built-in software. A region of interest was drawn and copied to the cross-section of each sample. The average and standard deviation were calculated.

With the addition of methylene blue, the OSCS, DS, or CS contaminated heparin (5 wt. %) had a stronger photoacoustic signal than pure heparin from 680 to 750 nm. While neither pure OSCS, DS, nor CS can enhance photoacoustic signal when methylene blue was added, additional OSCS in methylene blue can increase 7.5-fold signal increase compared to pure methylene blue and shows a characteristic peak at 715 nm. This peak is amplified compared to pure heparin in methylene blue (400% for 5 wt. % OSCS, 475% for 5 wt. % DS, and 476% for 5 wt. % CS). The photoacoustic signal of the contaminated heparin drops to background at 850 nm. These are favorable features to differentiate contaminated heparin from pure heparin. Additionally, the photoacoustic signals of the three contaminated heparin are stable (less spikes) at the wavelengths after 730 nm. A stable signal can help increase the accuracy of detection and lower the limit of detection of the specimen of interest.

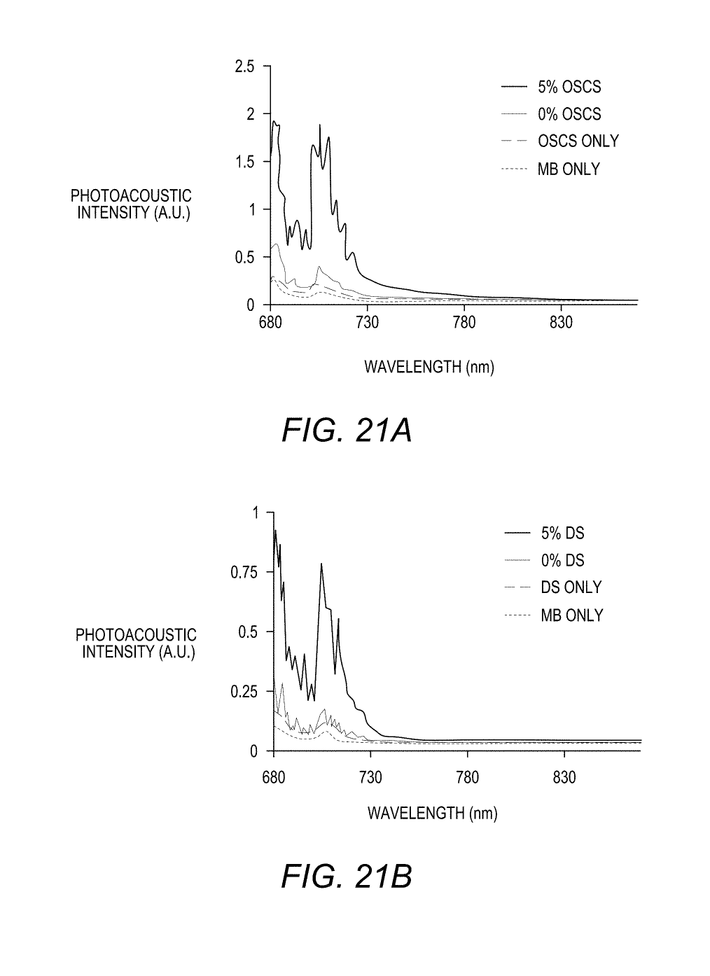

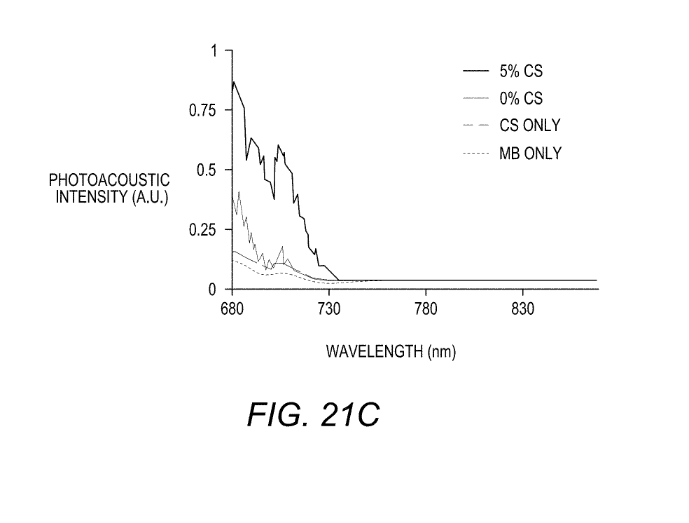

FIGS. 21A, 21B, and 21C show photoacoustic spectra of 5 wt. % OSCS (A), DS (B), and CS (C) contaminated heparin (red) in the presence of methylene blue. Controls include pure heparin (0% OSCS, DS, or CS; blue), contaminant only (OSCS, DS, or CS only; grey), and pure methylene blue (black). The contaminated heparin (red) has a much higher signal and a different spectral fingerprint than pure heparin (blue). Note also that this technique can discriminate between different types of contamination, e.g., OSCS (A) versus CS (C). Photoacoustic measurements of the same materials may also be employed to discriminate between different amounts of heparin contamination. The detection limit of OSCS in heparin is 0.3 ug/mL (or 0.03%) at 750 nm.

Thus, systems and methods according to present principles provide a fast imaging technique to quickly differentiate OSCS or DS contaminated heparin from heparin. The technique has a competitive detection limit and very fast analysis time (20 measurements per second). The detection limits are lower than the recommended standard of 0.1%. This approach could be used to monitor heparin in real time in a manufacturing process or could be used to diagnose a patient showing symptoms of exposure to contaminated heparin.

Other variations will also be understood.

1. A device for noninvasively monitoring therapy, comprising:
a. a source of photoacoustically active dye, the dye photoacoustically active at least when bonded to a quantity of interest;
b. an ultrasound photoacoustic imager configured to detect the level of the quantity of interest, the quantity of interest having been bonded to the dye,
c. such that the level of the quantity of interest in a patient undergoing therapy can be determined and maintained within a therapeutic range.

2. The device of claim 1, wherein the quantity of interest is heparin.

3. The device of claim 2, wherein the dye is a charged species that interacts with heparin.

4. The device of claim 3, wherein the charged species that interacts with heparin is a type of methylene blue.

5. The device of claim 3, wherein the dye is a phenothiazinium species selected from the group consisting of: toluidine blue, phenothiazinium dye, methyl methylene blue, dimethyl methylene blue, azure B, azure C, thionin, methylene violet, and combinations of the above.

6. The device of claim 3, further comprising a source of protamine sulfate, such that if the level of heparin is determined to exceed the therapeutic range, the source of protamine sulfate is controlled to deliver protamine sulfate to the patient in a controlled manner.

7. The device of claim 1, wherein the quantity of interest is digoxin or phenytoin.

8. The device of claim 1, wherein the source of dye is a localized region of dye on a catheter.

9. The device of claim 8, wherein the source of dye is localized by disposing bound nanoparticles coupled to the dye on a desired region of the catheter.

10. The device of claim 9, wherein the nanoparticles are silica, poly(lactic-co-glycolic acid) nanoparticles, polymeric micelles.

11. The device of claim 10, wherein the ratio of dye to nanoparticles is 0.5 to 5 mg of dye per mL of nanoparticle volume.

12. The device of claim 8, wherein the source of dye is localized by covalently binding dye to the catheter.

13. The device of claim 8, wherein the localized region of dye on a catheter is a cladding region.

14. The device of claim 13, wherein the catheter further is configured to include a drug release region.

15. The device of claim 14, wherein the catheter is configured such that the cladding region is upstream of the drug release region when the catheter is inserted in a blood vessel.

16. The device of claim 1, wherein the source of dye is a localized cladding region of dye on an interior surface of bypass/ECMO tubing.

17. The device of claim 1, wherein the source of dye includes multiple localized cladding regions of dye, each with a different dye concentration, as part of a medical blood drop and dipstick test.

18. The device of claim 1, wherein the ultrasound photoacoustic imager includes a wearable transducer.

19. A method for monitoring a drug therapy, comprising:
a. administering a source of photoacoustically active dye to a patient, the dye photoacoustically active when bonded to an administered medicament within the patient;
b. performing ultrasound photoacoustic imaging of the patient, the imaging configured to measure a quantitative level of the medicament in the patient by measuring the photoacoustic signal from the bonded dye; and
c. providing an output of a measurement of the quantitative level of the medicament as determined by the photoacoustic imaging.

20. The method of claim 19, further comprising ceasing, reducing, starting, or increasing administration of the medicament based on the measured quantitative level of the medicament in the patient.

21. The method of claim 20, wherein the output is used as an input to a controlling step, the controlling causing administration of the medicament to cease, reduce, start, or increase, so as to maintain the quantitative level of the medicament in the patient to within a therapeutic range or window.

22. The method of claim 21, where the controlling includes transmitting a signal to an infusion pump.

23. The method of claim 19, wherein the drug is heparin.

24. The method of claim 23, wherein the dye is a charged species that interacts with heparin.

25. The method of claim 24, wherein the charged species that interacts with heparin is methylene blue.

26. The device of claim 24, wherein the dye is a phenothiazinium species selected from the group consisting of: toluidine blue, phenothiazinium dye, methyl methylene blue, dimethyl methylene blue, azure B, azure C, thionin, methylene violet, and combinations of the above.

27. A method for monitoring a patient, comprising:
a. administering a source of photoacoustically active dye to a patient, the dye photoacoustically active when bonded to a quantity of interest within the patient;
b. performing ultrasound photoacoustic imaging of the patient, the imaging configured to detect a quantitative level of the quantity of interest in the patient by measurement of the photoacoustic signal from the bonded dye; and
c. providing an output of a measurement of the quantitative level of the quantity of interest as determined by the photoacoustic imaging.

28. The device of claim 1, wherein the quantity of interest is digoxin or phenytoin.

29. The device of claim 28, wherein the dye is an acid dye selected from the group including aniline derivatives.
